(19) United States
(12) Patent Application Publication
(10) Pub. No.: US 2015/0327794 A1
(43) Pub. Date: Nov. 19, 2015

(54) SYSTEM AND METHOD FOR DETECTING AND VISUALIZING LIVE KINETIC AND KINEMATIC DATA FOR THE MUSCULOSKELETAL SYSTEM

(71) Applicant: Umm Al-Qura University, Makkah (SA)

(72) Inventors: Mohamed Abdur Rahman, Makkah (SA); Ahmad Muaz Qamar-ul-Islam, Makkah (SA); Saleh Basalamah, Makkah (SA)

(73) Assignee: Umm Al-Qura University, Makkah (SA)

(21) Appl. No.: 14/277,089

(22) Filed: May 14, 2014

(51) Int. Cl. A61B 5/11; A61B 5/22; A61B 5/00

(52) U.S. Cl. CPC A61B 5/1114; A61B 5/1118; A61B 5/744; A61B 5/0022; A61B 5/7465; A61B 5/4528; A61B 5/224; A61B 5/486; A61B 5/0004; A61B 2503/06; A61B 2560/0475

(57) ABSTRACT

A system and method using a single 3D motion sensing Microsoft Kinect for Windows augmented with computer vision algorithms for detecting, recognizing and tracking the movement of different joints of the body of a subject and deducing kinetic and kinematic data from those movements. The method requires only a single motion sensing device and is non-invasive as the subject does not need to wear any external devices on the body. The proposed method incorporates Second Life serious game environment where the patient, the therapist and other members of the community of interest can log-in any time and see all the subject's movements performed by an avatar in Second Life, thereby making the invention particularly useful for disabled hemiplegic children.

### FIELD OF THE INVENTION

This invention relates to a system and method for detecting and visualizing live kinetic and kinematic data for the musculoskeletal system. More particularly, the invention relates to a system and method using the Microsoft Kinect®^{a} camera for detecting and visualizing live kinetic and kinematic data for the musculoskeletal system for children with hemiplegia. ^{a} Kinect is a registered trademark owned by Microsoft Corporation of Redmond, Wash.

### BACKGROUND OF THE INVENTION

Human ailments and disabilities are treated with a myriad of diagnostic and treatment tools. Biofeedback is one such tool. Biofeedback was first introduced in the literature more than thirty years ago as a training tool used in rehabilitation settings to facilitate normal movement patterns after injury. Since then, biofeedback has been used primarily in rehabilitation settings, for example in the treatment of gait abnormalities in adults after stroke. Biofeedback also has been used to facilitate the normalization of gait patterns in children with cerebral palsy and in adults after amputation, after spinal cord injuries, after hip fractures, and after total hip and knee joint replacements.

Biofeedback is a technique that provides clinicians with a useful tool for designing a regimen of therapy and for giving clients instructions on how to modify movement patterns. Thus, biofeedback complements the already present internal feedback, i.e., visual, auditory, and proprioceptive feedback. Biofeedback typically is provided instantaneously to the learner, i.e., in real time, whereas other methods of external feedback, e.g., verbal and video feedback, are provided some time after the movement. More recently, a resurgence of interest in real-time feedback has developed because of the expansion of technology related to kinematic and kinetic biofeedback.

Kinematics is the branch of biomechanics concerned with the study of movement from a geometrical point of view. Kinetics is the branch of biomechanics concerned with what causes a body to move the way it does. Biomechanics researchers have available a wide array of equipment for studying human movement kinematics. Current kinetic and kinematic systems use sensors attached to the body to measure and understand many different aspects of human behavior. This has been particularly useful in treating stroke patients, rehabilitation, and understanding sedentary behavior.

When a segment of the human body moves, it rotates around an imaginary axis of rotation that passes through a joint to which it is attached. Movements of the human body are referenced to three imaginary cardinal planes, the sagittal plane, frontal plane, and transverse plane, with their respectively associated mediolateral, anteroposterior, and longitudinal axes. Most human movement is general motion, a complex combination of linear and angular motion components. Since linear and angular motions are “pure” forms of motion, it is sometimes useful to break complex movements down into their linear and angular components when performing an analysis.

The three imaginary cardinal planes bisect the mass of the body in three dimensions. The sagittal plane, also known as the anteroposterior (AP) plane, divides the body vertically into left and right halves, with each half containing the same mass. The frontal plane, also referred to as the coronal plane, splits the body vertically into front and back halves of equal mass. The horizontal or transverse plane separates the body into top and bottom halves of equal mass. For an individual standing in anatomical reference position, the three cardinal planes all intersect at a single point known as the body's center of mass or center of gravity. These imaginary reference planes exist only with respect to the human body. If a person turns at an angle to the right, the reference planes also turn at an angle to the right.

In accordance with conventional practice, biomechanists typically conduct quantitative analyses of human motion by adhering small, reflective markers over the subject's joint centers and other points of interest on the body, with marker locations depending on the purpose of the analysis, and using cameras to detect the positions of the markers during movement of the subject. Movement analysts today have quite an array of camera types from which to choose. The type of movement and the requirements of the analysis largely determine the camera and analysis system of choice. High-speed digital video cameras with infrared light rings encircling the lenses can capture high contrast images of the reflective markers. However, since most human movement is not constrained to a single plane it is typically necessary to use multiple cameras to ensure that all of the movements can be viewed and recorded accurately for a detailed analysis. Researchers typically position six to eight and sometimes more cameras around the staging area in strategic locations to enable generation of three-dimensional representations of the movements of the markers.

An important use of the kinetic and kinematic data obtained from such tools is in the design of appropriate and effective therapy. For instance, hemiplegia is a disability that renders half of a child's body immovable. Therapy includes exercises to move the affected joints and muscles. In order to provide quality of service, therapists need to know the certain kinetic as well as kinematic metrics. Conventional methods involve the attachment of devices to the patient's body, and/or the use of multiple cameras placed at different positions around the patient, leading to discomfort to the patient and increased complexity and cost of the diagnostic equipment.

A device that can detect movement without any direct connection between the subject and the device is Kinect, a line of motion sensing input devices originally introduced by Microsoft for games, and specifically for control of Xbox 360 and Xbox One video game consoles without the need for a game controller. A later development adapted the device for Windows PCs. The Kinect contains sensors that allow it to track the movement of objects and individuals in three dimensions and recognize the position in space of several body parts from a person standing or moving in front of it. Among the applications for Kinect is Video Kinect. This application can use Kinect's tracking functionality and Kinect sensor's motorized pivot to keep users in frame even as they move around.

Several non-game applications have been developed for the Kinect device. One such application is a video surveillance system that combines multiple Kinect devices to track groups of people even in complete darkness. Other non-game applications include presentation software that can be controlled by hand gestures, and a Kinect kiosk that can overlay a collection of dresses onto the live video feed of customers.

Researchers at the University of Minnesota have used Kinect to measure a range of disorder symptoms in children, creating new ways of objective evaluation to detect such conditions as autism, attention-deficit disorder and obsessive-compulsive disorder. Several groups have reported using Kinect for intraoperative review of medical imaging, allowing the surgeon to access the information without contamination.

In a paper titled “Towards Skeleton Biometric Identification Using the Microsoft Kinect Sensor”, researchers Ricardo M. Araujo, Gustavo Graña, and Virginia Andersson, all of the Federal University of Pelotas, Pelotas, RS, Brazil, discuss a passive biometric system in which the Kinect sensor extracts skeleton points from walking subjects and uses these points for biometric identification.

Applicants are not aware of any prior system and/or method that use the Microsoft Kinect device to detect, recognize and track the movement of different joints of the body, deduce kinetic and kinematic data from these movements, and then use that data to design a treatment protocol for the patient.

It would be desirable to have a diagnostic tool to detect, recognize and track the movement of different joints of the body and deduce kinetic and kinematic data from these movements without the need to wear any external devices on the body and without the need to use multiple cameras.

### SUMMARY OF THE INVENTION

The present invention provides a system and method to detect, recognize and track the movement of different joints of the body and deduce kinetic and kinematic data from these movements without the need to wear any external devices on the body and without the need to use multiple cameras.

The invention is a non-invasive system for detecting, recognizing and tracking the movement of different joints of a subject's body and deducing kinetic and kinematic data from those movements, comprising a single remote 3D motion sensing device that recognizes and tracks the movement of different joints of a subject's body, wherein data collected by the motion sensing device is fed to a Second Life®^{b} serious game environment, an online 3D virtual world developed by Linden Research, Inc. of San Francisco, Calif., where the subject, a therapist and other members of a community of interest can log-in any time and see all the subject's movements performed by an avatar in Second Life®. The motion sensing device is the 3D depth sensing Microsoft Kinect device. Software written by applicants captures frame data from the Kinect and detects patterns of change in the position of joints between different frames. For example, it can measure the linear displacement of a joint over a given period of time or the angular displacement of a joint with respect to another joint. From this data, the ability of the patient to move different parts of his/her body can be inferred. This kind of kinetic/kinematic data collected over a period of time can be plotted to see the improvement in the patient's movements. ^{b} Second Life is a registered trademark belonging to Linden Research, Inc. of San Francisco, Calif.

The invention feeds the data to a customized Second Life® viewer that moves an avatar on the screen according to the movements of the subject. The data file can also be stored and re-played offline at a later time. Since the data is stored in the cloud, the patient, the therapist and other members of the community of interest can log-in any time and see all the movements performed by an avatar in Second Life®. Storing joint data instead of an actual video saves a lot of space as well as bandwidth when playing back since the material of interest is the patient's movements and not his/her looks. From applicant's research, it has been found that games specially developed for children with disability, often called serious games, makes them more engaged with the therapy environment. The games developed in Second Life® give the children motivation to do those therapeutic actions that will provide required quality of improvement metrics to the therapist.

Hemiplegia is a disability that renders half of a child's body immovable. Therapy includes exercises to move the affected joints and muscles. In order to provide appropriate therapy and quality of service, therapists need to know the certain kinetic as well as kinematic metrics which require the use of certain devices that need to be brought in contact with the child's body.

The invention will now be described in connection with the accompanying drawings.

### BRIEF DESCRIPTION OF THE DRAWINGS

FIG. 1 is a block diagram of the overall multi-sensor, multimedia therapy environment.

FIG. 2 shows the candidate domains needed during modeling of a therapy database.

FIG. 3 is a schematic representation of three cardinal reference planes, the frontal plane, the sagittal plane and the transverse plane, and their relationship to the mediolateral axis (X axis), the longitudinal axis (Y axis) and the anteroposterior axis (Z axis).

### DESCRIPTION OF THE PREFERRED EMBODIMENT OF THE INVENTION

This invention uses the 3D depth sensing Microsoft Kinect device for Windows to detect, recognize and track the movement of different joints of the body and deduce kinetic and kinematic data from these movements. The method is non-invasive as the subject does not need to wear any external devices on the body. Further, the system and method of the invention uses a single 3D camera to detect motion in three dimensions. As contemplated herein the Kinect device is used to track movement of a child suffering from Hemiplegia, a disability that renders half of a child's body immovable. The proposed method incorporates Second Life® serious game environment where the live therapeutic movements of the child are synchronized between the physical and virtual world so that the patient, the therapist and other members of the community of interest can log-in any time and see all the movements performed by an avatar in Second Life®.

The invention incorporates two types of visualization interfaces. Firstly, Second Life® is used to show live physical world activity into the 3D virtual world, which acts as a serious game environment. Secondly, 2D interfaces have been developed to show analytical output where live plotting of different quality of performance metrics is shown. The invention assumes that both the therapist and the Hemiplegic child use Kinect camera to either record or playback the therapy sessions. The session can be controlled by a menu driven interface as well as a speech-based interface.

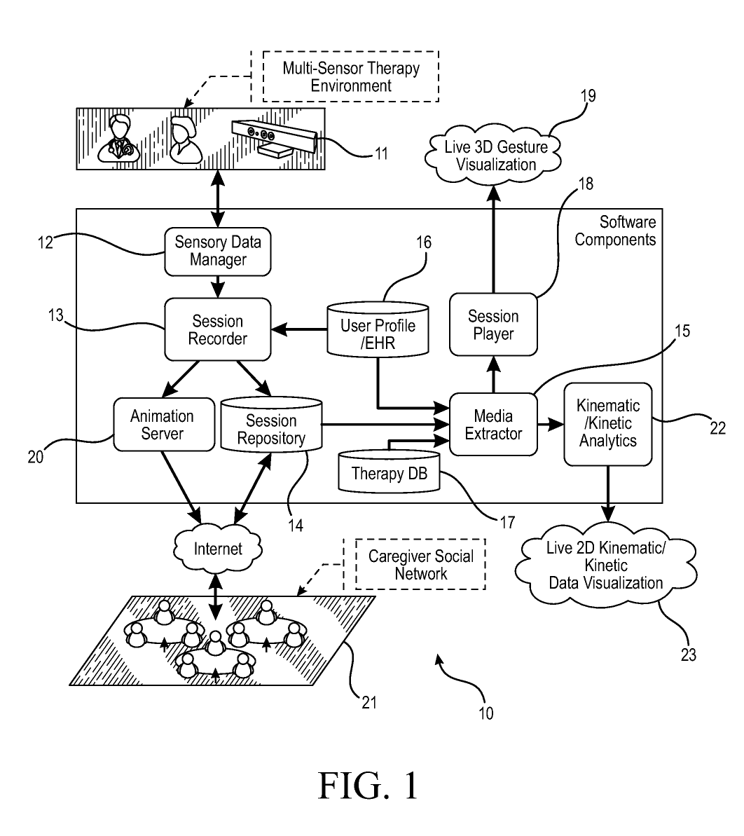

A block diagram of the overall multi-sensor multimedia therapy environment of the invention is indicated generally at 10 in FIG. 1. In the proposed system, applicants assume three types of users: disabled children; therapists; and caregivers such as parents. The system uses a single Kinect device 11 connected with a sensory data manager 12 that processes the raw data stream coming from the Kinect device and extracts joint data from the input. The data set contains the locations of twenty body joints observed thirty times per second. This component actually follows the model-view-controller (MVC) pattern.

Data from the sensory data manager 12 is fed to a session recorder 13 that records the exercise session which can then be saved to a user file through a session repository 14 and forwarded to a media extractor 15 for live-view in Second Life® or live plotting. The media extractor 15 extracts session data, combines it with user preferences from a user profile database 16 and a therapy database 17, both described below, and forwards it to a session player 18 that manages the movement of the avatar in the Second Life 3D gesture visualization interface 19. The data can also be sent by the session recorder 13 via network to an animation server 20 that can play the session on a remote system running the Second Life® viewer. The session recorder 13 provides options such as which joints need to be tracked and also displays graphs for those joints on the screen in real-time.

The animation server 20 facilitates the transmission of the session data over the network for remote viewing through a Second Life viewer. Using the animation and relay server, the framework establishes a live collaborative session between the therapist, the disabled child and the caregiver social network 21.

The session repository 14 stores the session data to secondary storage such as in the cloud so that it can be played back later in Second Life®. The session repository can also be accessed securely by authenticated community of interest (COI) for online electronic health record sharing purposes. COI, also referred to as caregiver social network 21, is a subset of social ties of a patient who gives medical services to him/her. An example of caregiver social network might be a patient's parents, family members, relatives, friends, medical staff in the therapy center or hospital, etc. Members of COI are part of a patient's user profile since they fall within the therapy lifecycle of the patient. For example, some members might be helping him/her in doing therapy at home; some might be helping in doing therapy sessions at the therapy center; and some might be carrying him/her to the center, to name a few.

The user profile database 16 is connected with the session recorder 13 and the media extractor 15. The user profile database acts as an electronic health record (EHR), which is used to store detailed information about the disabled child, the therapist and the caregiver. An example of a child record is family information, type of disability the child has, name of the therapist who is overseeing, types of therapy the child has to conduct, past history of therapy, recorded sessions, improvement parameters, etc.

The therapy database 17 connected with the media extractor 15 stores details about disability type, therapy types, types of motions involved in each therapy type, joints and muscles to be tracked in each motion type, metrics that store those joint and muscle values, normal ranges of each of the factors and metrics, and improvement metrics for each disability type, to name a few.

A kinematic/kinetic analytics component 22 connected with the media extractor 15 employs analytics and algorithms to provide live 2D kinematic/kinetic data visualization 23 of different quality of improvement metrics of different joints of a body analyzed from session data. This component is the heart of the whole framework. The visualization interfaces 19 and 23 show the joint positions over the course of a therapy module to output live graphs of joint positions after a session is complete. The visualization interface takes care of the starting and ending points of a particular session. For example, Algorithm1, below, shows details about how Kinematic Data Analysis takes place within the framework.

Algorithm 1: Kinematic Analysis of Live 2D Graph

```
View-Plot (SessionURI, PatientID, TherapyID)
begin
  View patient profile using PatientID;
  View available therapy sessions from online repository using SessionURI;
  Import a sessionXML file from SessionURI;
  If (sessionXML isReachable) then
    Load sessionXML file into Kinematic Analytics module;
    Load available APIs to access a particular therapy module;
    Find motion-types to be extracted from the therapy session;
    Foreach motionID {
      Find joint-types to be tracked;
      Call appropriate API to extract Flexion, Extension, Adduction and Abduction. . .
      movement data from the sessionXML file;
    }
  Show the set of available 2D graphs of different metrics based on analyzed motion
  and joint data;
end
```

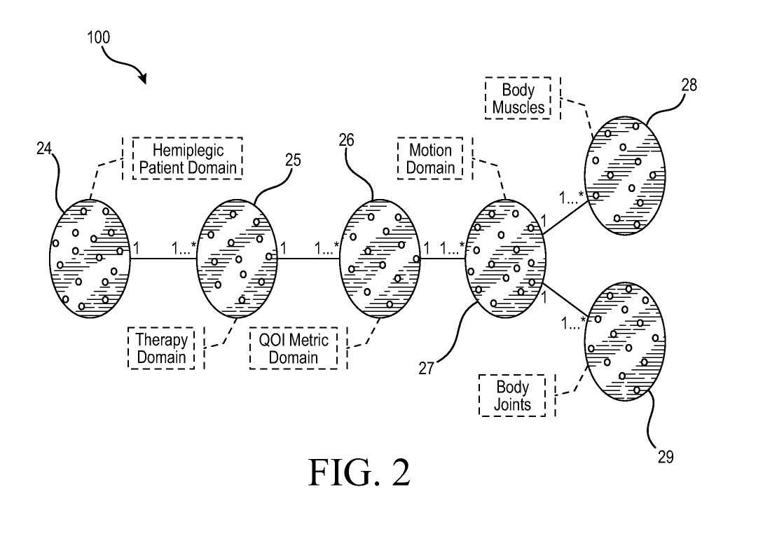

FIG. 2 shows a model 100 where each entity is connected to different other entities in a database. The patient domain 24 refers to the set of disabled person in our system. Each disabled person is assigned to one or more therapy modules from the therapy domain 25, which is the set of therapies available to the system. Each therapy is mapped to one or more quality of improvement metrics 26. Each metric is composed of variables in terms of different therapeutic motions that are mapped to a subset of motions from the motion domain 27. Each motion is composed of a subset of body muscles and joints that are mapped to the muscles and joints domains 28 and 29, respectively.

The software environment is set up such that a therapist can record live 3D gesture visualization of an exercise session in the 3D Second Life environment. Since Second Life supports a huge number of online participants at the same time and the same virtual space, the session can then be transmitted live over the network or uploaded to a virtual rehabilitation center developed inside the Second Life virtual world. The virtual world center inside Second Life looks similar to the real center where the child goes for therapy or does therapy at his/her home. The child can log on to Second Life and visit the virtual center where the practice session is being played or has been made available for download. The child can then view the session being performed by an avatar on the screen. The system can record the child's session and send it to the therapist. Temporal data collected from a number of sessions over a long period can be used to monitor the effectiveness and progress of the rehabilitation process.

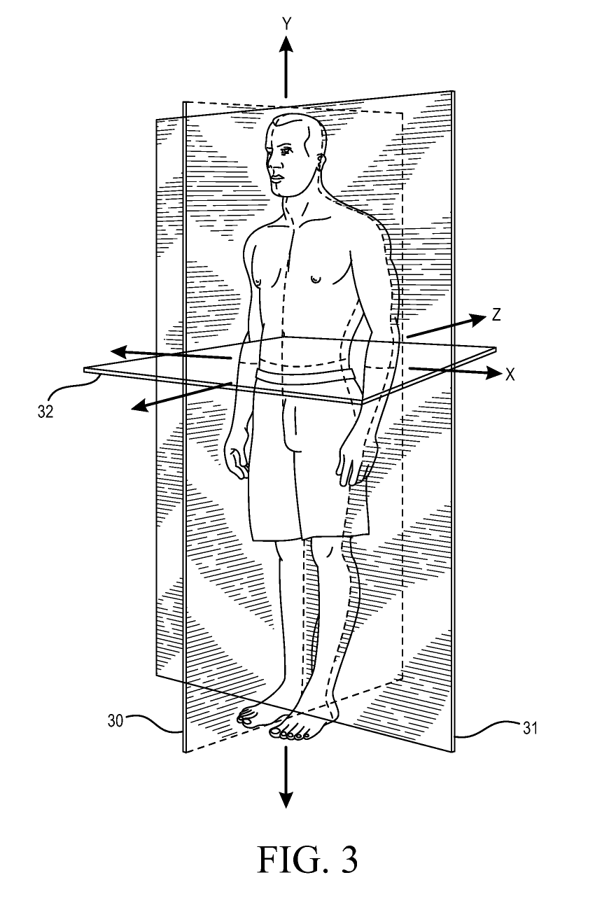

FIG. 3 shows the three imaginary cardinal reference planes that bisect the mass of the body in three dimensions. The sagittal plane 30, also known as the anteroposterior (AP) plane, divides the body vertically into left and right halves, with each half containing the same mass. The frontal plane 31, also referred to as the coronal plane, splits the body vertically into front and back halves of equal mass. The horizontal or transverse plane 32 separates the body into top and bottom halves of equal mass. For an individual standing in anatomical reference position, the three cardinal planes all intersect at a single point known as the body's center of mass or center of gravity. These imaginary reference planes exist only with respect to the human body. If a person turns at an angle to the right, the reference planes also turn at an angle to the right.

The present invention provides kinetic data such as muscle power, masses of each body segment with respect to the whole body mass, moments of inertia, and their locations, forces and moments of force at different joints, height of a person, etc. Prior to the present invention, this was not possible using non-invasive equipment.

The invention detects patterns of kinematic movement of the joints between different frames. This in turn helps in measuring the ability of the patient to move different parts of his/her body. Using joint angles of twenty body joints the system of the invention can track seventeen different therapeutic motions that take place due to gestures of different body parts and joints at any given time.

These personalized kinetic as well as kinematic data will provide a detailed analysis of the current state of the Hemiplegic patients' improvement.

The present invention is a non-invasive device and hence can be used with disabled hemiplegic children. Conventional complex measurement devices are bulky and restrict movements for even the normal child.

With a single 3D camera the invention enables the following motions around major joints to be detected and plotted in real time:

Abduction/Adduction

Flexion/Extension

Pronation/Supination

Inversion/Eversion

Hyperextension

Dorsiflexion/Plantar flexion

Rotation/Circumduction

Protraction/Retraction

Elevation/Depression

The above motions can be relayed live to the 3D virtual world. The terms used have their usual and customary meaning as given, for example, in “Kinematic Concepts for Analyzing Human Motion”, Chapter 2, Basic Biomechanics, 6^{th} edition, Susan J. Hall, Ph.D., 2012 McGraw-Hill Higher Education.

With a single 3D camera augmented with computer vision algorithms, the invention provides the following quality of information metrics for clinical data analysis:

Speed of movement

Force exerted by a muscle connected to a joint

Length of the bone connecting joints

Power of a muscle

Muscle moment

Force produced by a muscle

Torque developed by a joint

Subject height

Using novel clinical data analytics according to the invention, a therapist can visualize live statistical analysis of the above mentioned kinetic and kinematic motions and metrics and decide clinically the quality of improvement of a particular patient.

While the invention has been disclosed in connection with the preferred embodiments it should be recognized that changes and modifications may be made therein without departing from the scope of the appended claims.

What is claimed is:

1. A non-invasive system for detecting, recognizing and tracking the movement of different joints of a subject's body and deducing kinetic and kinematic data from those movements, comprising:
a single remote 3D motion sensing device that recognizes and tracks the movement of different joints of a subject's body; and
an online 3D virtual world serious game environment connected to receive data collected by the motion sensing device so that the subject, a therapist and other members of a community of interest can log-in any time and see all the subject's movements performed by an avatar in the virtual world game environment.

2. The system as claimed in claim 1, wherein:
the system includes a session recorder to record data collected by the motion sensing device.

3. The system as claimed in claim 2, wherein:
the system includes a session repository that stores the data in secondary storage where it can be played back later in the virtual world serious game.

4. The system as claimed in claim 3, wherein:
the system includes means storing a user profile that stores information about the subject, a therapist, and caregivers, wherein the information about the subject includes family information, type of disability the subject has, name of the therapist overseeing treatment, types of therapy the subject has to conduct, past history of therapy, recorded sessions, and improvement parameters.

5. The system as claimed in claim 4, wherein:
the system includes a therapy database that stores, but is not limited to, details about disability type, therapy types, types of motions involved in each therapy type, joints and muscles to be tracked in each motion type, metrics that store those joint and muscle values, normal ranges of each of the factors and metrics, and improvement metrics for each disability type.

6. The system as claimed in claim 5, wherein:
the system includes kinematic and kinetic analytics that provide live visualization of different quality of improvement metrics of different joints of a body analyzed from session data.

7. The system as claimed in claim 6, wherein:
the system includes a live 2D kinematic data visualization interface that receives information from the kinematic and kinetic analytics to show the subject's joint positions over the course of a therapy module to output live graphs of joint positions after a session is complete, said visualization interface noting the starting and ending point of a particular session.

8. The system as claimed in claim 7, wherein:
the system includes a session player interface that manages 3D movement of an avatar in the virtual world serious game, said movement of the avatar mimicking the movement of the subject.

9. The system as claimed in claim 8, wherein:
the system includes a media extractor connected with the session repository and the user profile, wherein the media extractor extracts session data, combines it with user preferences from the user profile database and the therapy database and forwards it to the session player.

10. The system as claimed in claim 9, wherein:
the system includes an animation server that facilitates transmission of session data over a network for remote viewing through the 3D virtual world serious game, establishing a live collaborative session between a therapist, a disabled subject and a caregiver social network.

11. A method for detecting, recognizing and tracking in three dimensions the movement of different joints of the body of a subject and obtaining kinetic and kinematic data from those movements, comprising the steps of:
using a single 3D motion sensing device to detect, recognize and track the movements;
capturing frame data from the motion sensing device and detecting patterns of change in the position of joints between different frames;
from said data determining the ability of the subject to move different parts of the body; and
plotting kinetic and kinematic data collected over a period of time to show improvement in the subject's movements.

12. The method as claimed in claim 11, including the step of:
feeding data collected by the motion sensing device to an online 3D virtual world serious game environment so that the subject, a therapist and other members of a community of interest can log-in any time and see all the subject's movements performed by an avatar in the virtual world game environment.

13. The method as claimed in claim 12, including the steps of:
recording in a session recorder data collected by the motion sensing device.

14. The method as claimed in claim 13, including the step of:
providing a user profile that includes information about the subject, a therapist, and caregivers, wherein the information about the subject includes family information, type of disability the subject has, name of the therapist overseeing treatment, types of therapy the subject has to conduct, past history of therapy, recorded sessions, and improvement parameters; and
supplying said information to the session recorder to determine which joints need to be tracked and displaying graphs for those joints on a screen in real-time.

15. The method as claimed in claim 14, including the steps of:
providing a therapy database that contains information about disability type, therapy types, types of motions involved in each therapy type, joints and muscles to be tracked in each motion type, metrics that store those joint and muscle values, normal ranges of each of the factors and metrics, and improvement metrics for each disability type.

16. A method for detecting, recognizing and tracking in three dimensions the movement of different joints of the body of a disabled patient and deducing kinetic and kinematic data from those movements, comprising the steps of:
viewing a patient profile containing an electronic health record storing detailed information about the disabled patient, a therapist and a caregiver, said information including but not limited to family information, type of disability the patient has, name of the therapist who is overseeing, types of therapy the patient has to conduct, past history of therapy, recorded sessions, and improvement parameters;
viewing available therapy sessions from an online repository that stores session data;
importing a session XML file;
if the session XML file is reachable, then:
loading the session XML file into a kinematic analytics module;
load available APIs to access a particular therapy module;
identify motion types to be extracted from the therapy session;
for each motion ID:
identify joint types to be tracked;
call an appropriate API to extract flexion, extension, adduction and
abduction movement data from the session XML file; and
displaying a set of available 2D graphs of different metrics based on the analyzed motion and joint data.

17. A method for detecting, recognizing and tracking in three dimensions the movement of different joints of the body of a child disabled with hemiplegia and obtaining kinetic and kinematic data from those movements, comprising the steps of:
using a single 3D motion detecting device to detect and plot in real time data relating to the following motions around major joints: abduction and adduction; flexion and extension; pronation and supination; inversion and eversion; hyperextension; dorsiflexion and plantar flexion; rotation and circumduction; protraction and retraction; and elevation and depression; and
relaying said motions live to a 3d virtual world serious game environment where the child, a therapist and a caregiver can view in real time the therapeutic movement of the child.

18. The method as claimed in claim 17, wherein:
information kinetic and kinematic metrics including speed of movement, force exerted by a muscle connected to a joint, length of the bone connecting joints, power of a muscle, muscle movement, force produced by a muscle, torque developed by a joint, and subject height are provided for clinical data analysis, whereby a therapist can determine live statistical analysis of the kinetic and kinematic motions and metrics and decide clinically the quality of improvement of a particular patient.
